(19) United States
(12) Patent Application Publication
(10) Pub. No.: US 2019/0000727 A1
(43) Pub. Date: Jan. 3, 2019

(54) MAKEUP COSMETIC

(71) Applicant: SHISEIDO COMPANY, LTD., Tokyo (JP)

(72) Inventors: Malyn CONCINA, East Windsor, NJ (US); Akane YANAGIHARA, YOKOHAMA-SHI (JP)

(73) Assignee: SHISEIDO COMPANY, LTD., Tokyo (JP)

(21) Appl. No.: 16/021,439

(22) Filed: Jun. 28, 2018

Related U.S. Application Data
(60) Provisional application No. 62/526,493, filed on Jun. 29, 2017.

(51) Int. Cl. A61K 8/06; A61Q 1/02

(52) U.S. Cl. CPC A61K 8/066; A61Q 1/02

(57) ABSTRACT

The present invention provides a makeup cosmetic and method of manufacturing the same attained by a water-in-oil emulsion cosmetic supplemented with an oil-in-water emulsion as a water phase in which fine (nano-sized) oil droplets containing a solid oil and having a specified predetermined particle size are dispersed. Specifically, the present invention provides a makeup cosmetic comprising an oil-in-water-in-oil (O/W/O) composite emulsion in which an oil-in-water (O/W) emulsion is dispersed in an external oil phase, wherein the oil-in-water (O/W) emulsion comprises oil droplets having a number-average particle size of 200 nm or smaller and containing a solidified oil.

### CROSS REFERENCE TO RELATED APPLICATIONS

This application relates to, and claims priority from, U.S. Prov. Ser. No. 62/526,493 filed Jun. 29, 2017, the entire contents of which are incorporated herein by reference.

### FIGURE SELECTED FOR PUBLICATION

FIG. 1

### BACKGROUND OF THE INVENTION

### Field of the Invention

The present invention relates to a makeup cosmetic and method of making the same disclosed herein. More specifically, the present invention relates to a makeup cosmetic in the form of an oil-in-water-in-oil (O/W/O) composite emulsion and having an internal oil phase consisting of fine (nano-sized) oil droplets containing a solid oil. The makeup cosmetic of the present invention offers a favorable texture upon application and is excellent not only in long lasting effect of a makeup but also provides a beneficial skincare.

### Description of the Related Art

Makeup cosmetics are mainly aimed at producing a beauty effect by, for example, covering up skin blemish or putting beautiful color on the skin. The makeup cosmetics may exert a psychological effect in such a way that makeup positively motivates people. Also, the makeup cosmetics, which stay on the skin for a long time, are naturally required to be not harmful to the skin. Needless to say, the makeup cosmetics preferably have a skincare effect of actively protecting the skin or improving skin texture.

The makeup cosmetics are, in general, rich in powder ingredients such as pigments and oily ingredients such as emollients, as compared with skincare cosmetics. Thus, the makeup cosmetics are often provided in the form of oil-based cosmetics or water-in-oil emulsion cosmetics. The water-in-oil emulsion cosmetics have such essential problems that the cosmetics offer an oily feeling after application and poorly hydrate the skin, because their external phases are oil phases. In addition, the emulsion stability of the water-in-oil emulsion cosmetics is more difficult to maintain than that of oil-in-water emulsion cosmetics (see JP-A 2008-24630).

On the other hand, most of moisturizing ingredients such as polyhydric alcohols are water-soluble or hydrophilic. A water-in-oil emulsion cosmetic containing a large amount of such a moisturizing ingredient might impair emulsion stability. JP-A 2003-155216 states that an alkylene oxide derivative having a specific structure can impart a moisturizing effect to a water-in-oil emulsion cosmetic superior to that of conventional moisturizing ingredients such as polyhydric alcohols. However, the technique of JP-A 2003-155216 is exceedingly limited by the structure of the alkylene oxide derivative that exerts a moisturizing effect, and cannot solve the problem of a feeling after application originally possessed by water-in-oil emulsion cosmetics.

JP-A 2008-24630 proposes that a water-in-oil emulsion makeup cosmetic produces a hydrated feeling after application by increasing the amount of an internal water phase by use of a specific surfactant, and further proposes to suppress moisture loss from the skin by adding a solid oil to an external oil phase. However, the technique of JP-A 2008-24630 is also limited by the mixing ratios of the surfactant and each ingredient for maintaining the stability of the system and therefore has very narrow options of formulas. Furthermore, the addition of a solid oil to an external oil phase disadvantageously reduces the spread of the cosmetic.

Accordingly, an object of the present invention is to provide a makeup cosmetic that is excellent in long lasting finish of makeup and in texture upon application and further has an excellent skincare effect.

### ASPECTS AND SUMMARY OF THE INVENTION

The present inventor has conducted extensive studies to attain the object and consequently completed the present invention by surprisingly finding that the object can be attained by a water-in-oil emulsion cosmetic supplemented with an oil-in-water emulsion as a water phase in which fine (nano-sized) oil droplets containing a solid oil and having a specified predetermined particle size are dispersed.

Specifically, the present invention provides a makeup cosmetic comprising an oil-in-water-in-oil (O/W/O) composite emulsion in which an oil-in-water (O/W) emulsion is dispersed in an external oil phase, wherein the oil-in-water (O/W) emulsion comprises oil droplets having a number-average particle size of 200 nm or smaller and containing a solid oil.

The makeup cosmetic of the present invention produces a hydrated feeling after application based on the internal water phase by emulsion break-up upon application to the skin. Furthermore, the solid oil contained in the internal oil phase spreads over the skin in collaboration with the oil in the external oil phase to form a protective covering containing the solid oil, thereby improving long lasting finish of makeup while suppressing moisture loss from the skin and exerting an excellent moisturizing effect to the skin. The makeup cosmetic of the present invention containing the solid oil in the internal oil phase spreads easily upon application to the skin and produces a unique pleasant feeling upon application.

It is generally known that the emulsion stability of water-in-oil emulsions having an internal phase containing an oil-in-water emulsion, i.e., oil-in-water-in-oil (O/W/O) composite emulsions, are more difficult to maintain than that of water-in-oil emulsions. For example, in order to stabilize an O/W/O composite emulsion, JP-A 2001-354515 proposes that an internal oil phase is included in cyclodextrin, and JP-A Hei 9-276676 proposes that organic modified clay mineral is added to an oil phase. However, it is an astonishing and surprising result and well beyond the prediction of those skilled in the art, that a stable O/W/O composite emulsion is obtained without stabilizing agent, such as cyclodextrin or organic modified clay mineral, by specifically controlling and adjusting the particle size of an internal oil phase containing a solid oil.

The present invention also enables a method of manufacturing the cosmetic according to the disclosure and details herein and below.

### BRIEF DESCRIPTION OF THE DRAWING

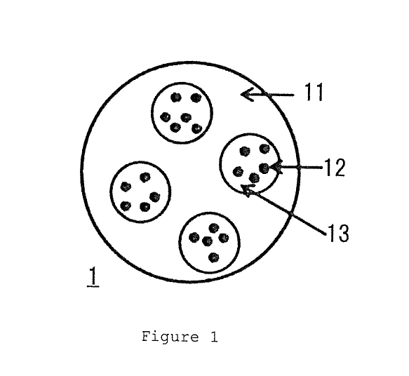

FIG. 1 is a schematic diagram showing the structure of the makeup cosmetic of the present invention.

### DETAILED DESCRIPTION OF THE PREFERRED EMBODIMENTS

The makeup cosmetic of the present invention is in the form of an oil-in-water-in-oil (O/W/O) composite emulsion (hereinafter, referred to as a “composite emulsion”). As shown in FIG. 1, this composite emulsion 1 has a structure where water phase 13 is dispersed in external oil phase 11 and internal oil phase 12 is dispersed in the water phase 13.

The water phase 13 in the cosmetic of the present invention is an oil-in-water emulsion. Specifically, the water phase 13 is an emulsion in which internal oil phase 12 is dispersed in the water phase.

The internal oil phase 12 forms oil droplets containing a solid oil.

In this context, the “solid oil” is an oily ingredient that is in a solid state at standard room temperature, i.e., 25° C. to 30° C.

The solid oil that can be used in the present invention can be any solid oil that can be usually added to cosmetics or the like. Examples thereof can include higher alcohols, solid fats and oils, waxes, solid hydrocarbons, higher fatty acids, and higher fatty acid salts. Among them, a higher alcohol, a higher fatty acid, or a higher fatty acid salt (particularly, an inorganic salt) is preferably used.

Examples of the higher alcohols include: linear higher alcohols such as lauryl alcohol, cetyl alcohol, stearyl alcohol, behenyl alcohol, myristyl alcohol, and cetostearyl alcohol; and branched higher alcohols such as glyceryl monostearyl ether (batyl alcohol) and glyceryl monocetyl ether (chimyl alcohol).

Examples of the higher fatty acids can include capric acid, lauric acid, myristic acid, palmitic acid, stearic acid, arachic acid, behenic acid, lignoceric acid, cerotinic acid, 2-palmitoleic acid, and erucic acid. Examples of the higher fatty acid salts can include: inorganic basic salts such as sodium salt, potassium salt, and ammonium salt; alkanolamine salts such as triethanolamine salt and tripropanolamine salt; metal salts such as aluminum salt, magnesium salt, zinc salt, and calcium salt; and basic amino acid salts such as arginine salt and lysine salt, of the higher fatty acids.

Lauric acid, myristic acid, palmitic acid, stearic acid, arachic acid, or behenic acid, or a salt thereof is particularly preferably contained in the emulsion composition of the present invention.

Examples of the solid fats and oils can include cacao butter, coconut oil, palm oil, palm kernel oil, hydrogenated oil, hydrogenated castor oil, Japan tallow, and shea butter. Examples of the waxes can include beeswax, carnauba wax, lanolin, reduced lanolin, hard lanolin, jojoba wax, and shellac wax.

Examples of the solid hydrocarbons can include Vaseline and microcrystalline wax.

The internal oil phase may contain, in addition to the solid oil, an additional oily ingredient (oil that is in a liquid state at room temperature (hereinafter, referred to as a “liquid oil”)) without inhibiting the effects of the present invention. The liquid oil that may be contained in the internal oil phase is not particularly limited and can be selected from liquid oils that can be added to cosmetics or the like.

The ratio between the solid oil and the liquid oil contained in the internal oil phase is not particularly limited as long as a water phase comprising a stable oil-in-water emulsion can be prepared. Usually, the internal oil phase preferably contains 10 to 95% by mass, preferably 20 to 70% by mass, more preferably 25 to 60% by mass of the solid oil with respect to the total amount of the internal oil phase.

In the cosmetic of the present invention, the internal oil phase containing the solid oil is oil droplets having a number-average particle size of 200 nm or smaller, preferably 100 nm or smaller, more preferably 50 nm or smaller. Surprisingly, if the number-average particle size of the droplets of this internal oil phase exceeds 200 nm, a unique hydrated feeling after application, which is a feature of the present invention, is not obtained. The lower limit value of the number-average particle size is not particularly limited and is usually 10 nm or larger, preferably 20 nm or larger, more preferably 30 nm or larger.

The average particle size of the oil droplets in the present description is considered the average value of the diameter of the oil droplets optically measured by the dynamic light scattering method or the like with an assumption that the particle shape of the oil droplets is spherical. Naturally, the average particle size of the oil droplets in the present disclosure can employ all the values between the upper value and the lower value described above.

The content of the internal oil phase is 1 to 40% by mass, preferably 3 to 30% by mass, more preferably 6 to 26% by mass, with respect to the total amount of the water phase. If the content is less than 1% by mass, the effects of the present invention cannot be exerted. When the content exceeds 40% by mass, it does not form O/W/O emulsion.

The ingredients constituting the oil-in-water emulsion which is a water phase are water and an optional aqueous ingredient, in addition to the internal oil phase (oil droplets containing a solid oil).

The optional aqueous ingredient is not particularly limited and can be any water-soluble or hydrophilic ingredient that can be added to cosmetics. For example, a surfactant and a moisturizing agent such as a polyhydric alcohol is preferably used.

The surfactant is preferably added in order to stably prepare the oil-in-water emulsion serving as a water phase. The surfactant used in the present invention is not particularly limited and can be selected from ionic surfactants including anionic, cationic, and amphoteric surfactants, and nonionic surfactants. Among them, an anionic or nonionic surfactant is preferred, and an anionic surfactant is particularly preferred, from the viewpoint of the stability of the system. Use of a surfactant capable of forming a gel (alpha-gel) with an amphoteric substance (such as higher alcohol and/or higher fatty acid) and water can further improve the stability of the 0/W emulsion. In such a case, it is preferable to adjust the ration of content of the surfactant and amphoteric substance to in the range of 1:5 to 1:10.

Examples of the anionic surfactant include: fatty acid soaps such as sodium laurate and sodium palmitate; higher alkyl sulfuric acid esters salts such as sodium lauryl sulfate and potassium lauryl sulfate; alkyl ether sulfuric acid ester salts such as POE triethanolamine lauryl sulfate and sodium POE lauryl sulfate; N-acyl sarcosine acids such as lauroyl sarcosine sodium; higher fatty acid amide sulfonates such as N-myristoyl-N-methyltaurine sodium, coconut oil fatty acid methyltaurine sodium, and lauryl methyltaurine sodium; phosphoric acid ester salts such as sodium POE oleyl ether phosphate and sodium POE stearyl ether phosphoric acid; sulfosuccinates such as sodium di-2-ethylhexylsulfosuccinate, sodium monolauroyl monoethanolamide polyoxyethylene sulfosuccinate, and sodium lauryl polypropylene glycol sulfosuccinate; alkylbenzenesulfonates such as sodium linear dodecylbenzenesulfonate, triethanolamine linear dodecylbenzenesulfonate, and sodium linear dodecylbenzenesulfonic acid; higher fatty acid ester sulfuric acid ester salts such as sodium hydrogenated coconut oil fatty acid glycerin sulfate; N-acyl glutamates such as monosodium N-lauroyl glutamate, disodium N-stearoyl glutamate, and monosodium N-myristoyl-L-glutamate; sulfated oils such as turkey-red oil; POE alkyl ether carboxylic acids; POE alkyl allyl ether carboxylates; α-olefin sulfonates; higher fatty acid ester sulfonates; secondary alcohol sulfuric acid ester salts; higher fatty acid alkylolamide sulfuric acid ester salts; sodium lauroyl monoethanolamide succinate; ditriethanolamine N-palmitoyl aspartate; and casein sodium. Among them, an N-acyl glutamate, for example, sodium N-stearoyl glutamate, is preferred.

Examples of the nonionic surfactant include: POE sorbitan fatty acid esters such as POE sorbitan monooleate, POE sorbitan monostearate, POE sorbitan monolaurate, and POE sorbitan tetraoleate; POE sorbitol fatty acid esters such as POE sorbitol monolaurate, POE sorbitol monooleate, POE sorbitol pentaoleate, and POE sorbitol monostearate; POE glycerin fatty acid esters such as POE glycerin monostearate, POE glycerin monoisostearate, and POE glycerin triisostearate; POE fatty acid esters such as POE monooleate, POE distearate, POE monodioleate, and ethylene glycol distearate); POE alkyl ethers such as POE lauryl ether, POE oleyl ether, POE stearyl ether, POE behenyl ether, POE 2-octyl dodecyl ether, and POE cholestanol ether; Pluronic type such as Pluronic; POE/POP alkyl ethers such as POE/POP cetyl ether, POE/POP 2-decyl tetradecyl ether, POE/POP monobutyl ether, POE/POP hydrogenated lanolin, and POE/POP glycerin ether; tetra-POE/tetra-POP ethylenediamine condensates such as Tetronic; POE castor oil or hydrogenated castor oil derivatives such as POE castor oil, POE hydrogenated castor oil, POE hydrogenated castor oil monoisostearate, POE hydrogenated castor oil triisostearate, POE hydrogenated castor oil monopyroglutamic acid monoisostearic acid diester, and POE hydrogenated castor oil maleic acid; POE beeswax or lanolin derivatives such as POE sorbitol beeswax; alkanolamides such as coconut oil fatty acid diethanolamide, lauric acid monoethanolamide, and fatty acid isopropanolamide; POE propylene glycol fatty acid esters; POE alkylamines; POE fatty acid amides; sucrose fatty acid esters; alkylethoxydimethylamine oxides; and trioleyl phosphoric acid.

Examples of the polyhydric alcohol include: dihydric alcohols such as ethylene glycol, propylene glycol, trimethylene glycol, 1,2-butylene glycol, 1,3-butylene glycol, tetramethylene glycol, 2,3-butylene glycol, pentamethylene glycol, 2-butene-1,4-diol, hexylene glycol, and octylene glycol; trihydric alcohols such as glycerin, trimethylolpropane, and 1,2,6-hexanetriol; tetrahydric alcohols such as pentaerythritol; pentahydric alcohols such as xylitol; hexahydric alcohols such as sorbitol and mannitol; polyhydric alcohol copolymers such as diethylene glycol, dipropylene glycol, triethylene glycol, polypropylene glycol, triglycerin, tetraglycerin, and polyglycerin; dihydric alcohol alkyl ethers such as ethylene glycol monomethyl ether, ethylene glycol monoethyl ether, ethylene glycol monobutyl ether, ethylene glycol monophenyl ether, ethylene glycol monohexyl ether, ethylene glycol mono-2-methyl hexyl ether, ethylene glycol isoamyl ether, ethylene glycol benzyl ether, ethylene glycol isopropyl ether, ethylene glycol dimethyl ether, ethylene glycol diethyl ether, and ethylene glycol dibutyl ether; dihydric alcohol alkyl ethers diethylene glycol monomethyl ether, diethylene glycol monoethyl ether, diethylene glycol monobutyl ether, diethylene glycol dimethyl ether, diethylene glycol diethyl ether, diethylene glycol dibutyl ether, diethylene glycol methyl ethyl ether, triethylene glycol monomethyl ether, triethylene glycol monoethyl ether, triethylene glycol monoethyl ether, propylene glycol monomethyl ether, propylene glycol monoethyl ether, dipropylene glycol methyl ether, dipropylene glycol ethyl ether, and dipropylene glycol butyl ether; and dihydric alcohol ether esters such as ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disuccinate, ethylene glycol monoethyl ether acetate, diethylene glycol monobutyl ether acetate, propylene glycol monomethyl ether acetate, propylene glycol monoethyl ether acetate, propylene glycol monopropyl ether acetate, and propylene glycol monophenyl ether acetate.

Other examples of the moisturizing agent include chondroitin sulfate, hyaluronic acid, mucoitin sulfate, colanic acid, atelocollagen, and cholesteryl-12-hydroxystearate. Sodium lactate, bile acid monosalt, dl-pyrrolidonecarboxylic acid monosalt, short-chain soluble collagen, Chestnut rose extracts, Achillea millefolium extracts, or the like can also be added without deleting the viscosity of a base.

It is preferable to add PEG/PPG-14/7 dimethylether in order to improve penetrating feeling into the skin. The content of PEG/PPG-14/7 dimethylether is about 0.001 to 0.1% by mass.

Other examples of the aqueous ingredient that can be contained in the water phase include water-soluble ingredients including: water-soluble active substances including vitamins such as vitamin B group, vitamin C group and derivatives thereof, pantothenic acid and derivatives thereof, and biotin; buffers such as sodium glutamate, arginine, aspartic acid, citric acid, tartaric acid, and lactic acid; chelating agents such as EDTA; water-soluble ultraviolet absorbers; and various dyes. Also, a lower alcohol having 6 or less carbon atoms, such as ethanol or isopropanol, may be added to the water phase.

The cosmetic of the present invention is a composite emulsion in which the oil-in-water emulsion described above has been emulsified as a water phase in an external oil phase.

The oily ingredient constituting the external oil phase of the composite emulsion of the present invention is preferably selected from liquid oils that are used in cosmetics or the like.

Examples of the liquid oil that can be contained in the external oil phase include: liquid fats and oils such as avocado oil, camellia oil, turtle oil, macadamia nut oil, corn oil, mink oil, olive oil, rapeseed oil, egg yolk oil, sesame oil, persic oil, wheat germ oil, sasanqua oil, castor oil, flaxseed oil, safflower oil, cottonseed oil, evening primrose oil, perilla oil, soybean oil, peanut oil, tea oil, kaya oil, rice bran oil, Chinese tung oil, Japanese tung oil, jojoba oil, germ oil, triglycerin, glycerin trioctanoate, and glycerin triisopalmitate; solid fats and oils such as cacao butter, coconut oil, horse fat, hydrogenated coconut oil, palm oil, beef tallow, mutton tallow, hydrogenated beef tallow, palm kernel oil, lard, beef bone fat, Japan wax kernel oil, hydrogenated oil, neat's foot oil, Japan wax, and hydrogenated castor oil; waxes such as beeswax, candelilla wax, cotton wax, carnauba wax, bayberry wax, insect wax, spermaceti, montan wax, bran wax, lanolin, kapok wax, lanolin acetate, liquid lanolin, sugarcane wax, lanolin fatty acid isopropyl, hexyl laurate, reduced lanolin, jojoba wax, hard lanolin, shellac wax, POE lanolin alcohol ether, POE lanolin alcohol acetate, lanolin fatty acid polyethylene glycol, and POE hydrogenated lanolin alcohol ether; hydrocarbons such as liquid paraffin, ozokerite, squalene, pristane, paraffin, ceresin, squalane, Vaseline, and microcrystalline wax; and synthetic esters such as isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkyl glycol monoisostearate, neopentyl glycol dicaprate, diisostearyl malate, glycerin di-2-heptylundecanoate, trimethylolpropane tri-2-ethylhexanoate, trimethylolpropane triisostearate, pentaerythritol tetra-2-ethylhexanoate, glycerin tri-2-ethylhexanoate, trimethylolpropane triisostearate, cetyl 2-ethylhexanoate, 2-ethylhexyl palmitate, glycerin trimyristate, glyceride tri-2-heptylundecanoate, castor oil fatty acid methyl ester, oleyl oleate, cetostearyl alcohol, acetoglyceride, 2-heptylundecyl palmitate, diisopropyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, ethyl acetate, butyl acetate, amyl acetate, and triethyl citrate.

Other examples thereof can include: chain polysiloxanes such as dimethylpolysiloxane, methylphenylpolysiloxane, and methyl hydrogen polysiloxane; cyclic polysiloxanes such as octamethylcyclotetrasiloxane, decamethylcyclopentasiloxane, dodecamethylcyclohexasiloxane, and tetramethyl tetrahydrogen cyclotetrasiloxane; and silicone oils including modified silicone oils such as amino-modified silicone oil, epoxy-modified silicone oil, epoxy/polyether-modified silicone oil, polyether-modified silicone oil, carboxy-modified silicone oil, alcohol-modified silicone oil, alkyl-modified silicone oil, ammonium salt-modified silicone oil, and fluorine-modified silicone oil.

The external oil phase can contain, in addition to the liquid oil described above, for example, a powder ingredient, an ultraviolet absorber, an antiseptic such as paraben, vitamins such as ubiquinone and vitamin P, a bactericide such as chlorhexidine hydrochloride, trichlorocarbanilide, or Irgasan DP300, and an agent such as dexamethasone acetate.

Examples of the powder ingredient include: inorganic powders such as talc, kaolin, mica, sericite, muscovite, biotite, phlogopite, synthetic mica, vermiculite, magnesium carbonate, calcium carbonate, aluminum silicate, barium silicate, calcium silicate, magnesium silicate, tungstic acid metal salts, magnesium, silica, zeolite, barium sulfate, calcined calcium sulfate, calcined gypsum, calcium phosphate, fluorapatite, hydroxyapatite, ceramic powders, and metallic soaps (zinc myristate, calcium palmitate, aluminum stearate, etc.); organic powders such as polyamide resin powders, polyethylene powders, polymethyl methacrylate powders, polystyrene powders, styrene-acrylic acid copolymer resin powders, benzoguanamine resin powders, polyethylene tetrafluoride powders, and cellulose powders; inorganic white pigments such as titanium dioxide and zinc oxide; inorganic red pigments such as iron oxide (colcothar) and iron titanate; inorganic brown pigments such as γ-iron oxide; inorganic yellow pigments such as yellow iron oxide and ocher; inorganic black pigments such as black iron oxide, carbon, and lower titanium oxide; inorganic violet pigments such as mango violet and cobalt violet; inorganic green pigments such as chromium oxide, chromium hydroxide, and cobalt titanate; inorganic blue pigments such as ultramarine blue and iron blue; pearl pigments such as titanium oxide-coated mica, titanium oxide-coated bismuth oxychloride, titanium oxide-coated talc, colored titanium oxide-coated mica, bismuth oxychloride, and argentine; metal powder pigments such as aluminum powders and copper powders; organic pigments such as Red No. 202, Red No. 205, Red No. 220, Red No. 228, Red No. 405, Orange No. 203, Orange No. 204, Yellow No. 205, Yellow No. 401, and Blue No. 404; organic pigments of zirconium, barium, and aluminum lake such as Red No. 3, Red No. 104, Red No. 227, Red No. 401, Orange No. 205, Yellow No. 4, Yellow No. 202, Green No. 3, and Blue No. 1; and natural dyes such as chlorophyll and β-carotene.

The content of the powder ingredients is not specifically limited. The content of powder ingredients is normally 1.0 to 50.0% by mass, preferably 5.0 to 40% by mass, and more preferably 7.0 to 30% by mass.

The cosmetic of the present invention can be prepared by preparing an oil-in-water emulsion constituting a water phase, and subsequently emulsifying the obtained oil-in-water emulsion into an external oil phase to prepare a composite emulsion.

The method for preparing the oil-in-water emulsion serving as a water phase is not particularly limited and can be any method to produce a stable emulsion. For example, high-pressure emulsification treatment using Nanomizer, Manton Gaulin, a French press, a colloid mill, or a microfluidizer, or ultrasonic emulsification treatment using an ultrasonic emulsifying machine is preferably used. Use of these treatments produces an oil-in-water emulsion in a cream form containing an internal-phase oil finely and stably and having a smooth appearance with gross, which is particularly preferred for the preparation of the composite emulsion of the present invention.

Subsequently, the obtained oil-in-water emulsion (water phase) is mixed and emulsified with stirring into an external-phase oil containing, if necessary, an emulsifier, to obtain the composite emulsion of the present invention. The emulsifying machine for use in this operation is not particularly limited and is preferably Disper (TK HOMD DISPER; Tokushu Kika Kogyo Co., Ltd.) or the like.

The number-average particle size of the water phase dispersed in the external oil phase is not particularly limited and is preferably 1 to 10 μm.

The external oil phase may contain an oil-soluble thickening agent such as organic modified clay mineral. On the other hand, it is possible to adjust viscosity of the makeup cosmetic of the present invention by changing content of water phase (0/W emulsion). Accordingly, the viscosity of the makeup cosmetic of the present invention is not specifically limited.

The makeup cosmetic of the present invention comprising the composite emulsion thus prepared offers hydrated texture upon application to the skin, and forms a protective covering on the skin by the solid oil contained in the internal oil phase after the application, thereby improving long lasting finish of makeup and a moisturizing effect, in spite of being an emulsion cosmetic having an oil phase as an external phase. Thus, the cosmetic of the present invention is particularly suitable as a cosmetic for base makeup such as a foundation.

### EXAMPLES

Hereinafter, the present invention, of both the cosmetic and the method of manufacturing the cosmetic, will be described in more detail with reference to Examples. However, the present invention is not intended to be limited by these Examples by any means. The content of each ingredient is indicated by % by mass with respect to the total amount of a composition containing the ingredient, unless otherwise specified.

O/W emulsion of each example having the formula shown in Table 1 below was prepared by a high-pressure emulsification using a Manton Gaulin. The viscosity and particle size of each example were measured, and appearance (transparency) and texture of each example were evaluated by a panel of experts.

TABLE 1

| | | Example | Example | Example | Example |
|---|---|---|---|---|---|
| Component | Ingredients | A | B | C | D |
| Water | Water | 47.34 | 47.4 | 44.96 | 48.19 |
| Humectant | Glycerin | 22 | 22 | 22 | 22 |
| | Butylene Glycol | 11 | 11 | 11 | 11 |
| | PEG/PPG-14/7 | 0.01 | 0.01 | 0.01 | 0.01 |
| | Dimethyl Ether | | | | |
| Surfactant | Sodium Stearoyl | 0.85 | 0 | 0 | 0 |
| | Glutamate | | | | |
| | Sodium Methyl | 0 | 0.79 | 0 | 0 |
| | Stearoyl Taurate | | | | |
| | Beheneth-30 | 0 | 0 | 3.23 | 0 |
| Oil | Hydrogenated | 7 | 7 | 7 | 7 |
| | Polydecene | | | | |
| | Pentaerythrityl | 2 | 2 | 2 | 2 |
| | Tetraethyl- | | | | |
| | hexanoate | | | | |
| | Stearic Acid | 1.3 | 1.3 | 1.3 | 1.3 |
| | Behenic Acid | 1.5 | 1.5 | 1.5 | 1.5 |
| | Stearyl Alcohol | 0.8 | 0.8 | 0.8 | 0.8 |
| | Behenyl Alcohol | 3.4 | 3.4 | 3.4 | 3.4 |
| | Dimethicone | 2.8 | 2.8 | 2.8 | 2.8 |
| Preservative | Phenoxyethanol | 0.5 | 0.5 | 0.5 | 0.5 |
| Neutralizing | Potassium | 0.4 | 0.4 | 0.4 | 0.4 |
| agent | Hydroxide | | | | |
| | Total | 100 | 100 | 100 | 100 |
| | VISCOSITY | 210 | 130 | 1550 | N/A |
| | (LVT Spindle #2, | | | | |
| | 12 rpm, 25 C.) | | | | |
| | Particle size (nm) | 42 | 35 | 83 | N/A |
| | APPEARANCE | 5 | 5 | 3 | 1 |
| | (TRANS- | | | | |
| | LUCENCY) | | | | |
| | TEXTURE | 3 | 3 | 3 | 1 |

As is shown from Table 1, O/W emulsions having an average particle size of 200 nm or less have transparent appearance and excellent texture. Examples A and B using anionic surfactants are superior in transparency to Example C using nonionic surfactant.

Each O/W/O composite emulsion (foundation) containing the O/W emulsion of Example A, B or C in its inner phase was prepared with the formula shown in Table 2 below. The viscosity of each composite emulsion was measured. The properties (RADIANCE, LONG WEAR & MAKEUP ADHESION, COVERAGE, SKINCARE BENEFITS (IMMEDIATE), SKINCARE BENEFITS (LONG TERM), and TEXTURE) of each composite emulsion was evaluated by a panel of experts, and rated them 5 (best) to 1 (worst).

TABLE 2

| | | Example | Example | Example |
|---|---|---|---|---|
| | Ingredients | 1 | 2 | 3 |
| OIL PHASE | Disteardimonium Hectorite | 0.4 | 0.4 | 0.4 |
| | Dimethicone | 17.5 | 17.5 | 17.5 |
| | Phenyl Trimethicone | 6 | 6 | 6 |
| | PPG-3 Benzyl Ether Myristate, | 2 | 2 | 2 |
| | Pentaerythrityl Tetra-Di-T-Butyl | | | |
| | Hydroxyhydrocinnamate | | | |
| | Trimethylsiloxysilicate, | 4 | 4 | 4 |
| | Cyclopentasiloxane | | | |
| | PEG-10 Dimethicone, Tocopherol | 2.6 | 2.6 | 2.6 |
| | Bis-ButylDimethicone | 1.5 | 1.5 | 1.5 |
| | Polyglyceryl-3 | | | |
| | Sorbitan Sesquiisostearate | 1 | 1 | 1 |
| | Polymethylsilsesquioxane | 3 | 3 | 3 |
| POWDER | Boron Nitride, Dimethicone | 2 | 2 | 2 |
| | Titanium Dioxide, Mica, Tin Oxide | 1.5 | 1.5 | 1.5 |
| | Mica, Barium Sulfate, Titanium | 1 | 1 | 1 |
| | Dioxide, Alumina | | | |
| | Titanium Dioxide, Aluminum | 13 | 13 | 13 |
| | Dimyristate, Triethoxycaprylylsilane, | | | |
| | Disodium Stearoyl Glutamate | | | |
| | Iron Oxide, Aluminum Dimyristate, | 7 | 7 | 7 |
| | Triethoxycaprylylsilane, Disodium | | | |
| | Stearoyl Glutamate | | | |
| TEXTURIZER | Dimethicone, Dimethicone | 15 | 15 | 15 |
| | Crosspolymer | | | |
| WATER | O/W EMULSION | Example A | Example B | Example C |
| PHASE | | 10 | 10 | 10 |
| | Glycerin | 1 | 1 | 1 |
| | Water | 11 | 11 | 11 |
| | Phenoxyethanol | 0.5 | 0.5 | 0.5 |
| | Total | 100 | 100 | 100 |
| PROPERTIES | VISCOSITY (LVT Spindle #4, | 10,000 cps | 13,500 cps | 13,500 cps |
| | 12 rpm, 25 C.) | | | |
| | RADIANCE | 5 | 5 | 5 |
| | LONG WEAR & MAKEUP | 5 | 5 | 5 |
| | ADHESION | | | |
| | COVERAGE | 5 | 5 | 4 |
| | SKINCARE BENEFITS | 5 | 5 | 5 |
| | (IMMEDIATE) | | | |
| | SKINCARE BENEFITS | 5 | 5 | 5 |
| | (LONG TERM) | | | |
| | TEXTURE | 5 | 4 | 4 |

All of the composite emulsions of Examples 1 to 3 in Table 2 had excellent properties. Among them, Example 1 containing an anionic surfactant, in particular, sodium Stearoyl Glutamate, had evaluation rate of “5 (best)” in all properties.

O/W/O composite emulsions with the formulas in Tables 3-1 and 3-2 below were prepared using O/W emulsion of Example A.

TABLE 3-1

| | | Example | Example | Example | Example |
|---|---|---|---|---|---|
| | Ingredients | 4 | 5 | 6 | 7 |
| OIL PHASE | Disteardimonium Hectorite | 0.4 | 0.4 | 0.4 | 0.4 |
| | Dimethicone | 17.5 | 17.5 | 17.5 | 17.5 |
| | Phenyl Trimethicone | 6 | 6 | 6 | 6 |
| | PPG-3 Benzyl Ether Myristate, | 2 | 2 | 2 | 2 |
| | Pentaerythrityl Tetra-Di-T-Butyl | | | | |
| | Hydroxyhydrocinnamate | | | | |
| | Trimethylsiloxysilicate, | 4 | 4 | 4 | 4 |
| | Cyclopentasiloxane | | | | |
| | PEG-10 Dimethicone, Tocopherol | 2.6 | 2.6 | 2.6 | 2.6 |
| | Bis-ButylDimethicone Polyglyceryl-3 | 1.5 | 1.5 | 1.5 | 1.5 |
| | Sorbitan Sesquiisostearate | 1 | 1 | 1 | 1 |
| | Polymethylsilsesquioxane | 3 | 3 | 3 | 3 |
| POWDER | Boron Nitride, Dimethicone | 2 | 2 | 2 | 2 |
| | Titanium Dioxide, Mica, Tin Oxide | 1.5 | 1.5 | 1.5 | 1.5 |
| | Mica, Barium Sulfate, Titanium | 1 | 1 | 1 | 1 |
| | Dioxide, Alumina | | | | |
| | Titanium Dioxide, Aluminum | 13 | 13 | 13 | 13 |
| | Dimyristate, Triethoxycaprylylsilane, | | | | |
| | Disodium Stearoyl Glutamate | | | | |
| | Iron Oxide, Aluminum Dimyristate, | 7 | 7 | 7 | 7 |
| | Triethoxycaprylylsilane, Disodium | | | | |
| | Stearoyl Glutamate | | | | |
| TEXTURIZER | Dimethicone, Dimethicone | 15 | 15 | 15 | 15 |
| | Crosspolymer | | | | |
| WATER | O/W EMULSION (Example A) | 1 | 5 | 10 | 15 |
| PHASE | Glycerin | 1 | 1 | 1 | 1 |
| | Water | 20 | 16 | 11 | 6 |
| | Phenoxyethanol | 0.5 | 0.5 | 0.5 | 0.5 |
| | Total | 100 | 100 | 100 | 100 |
| PROPERTIES | VISCOSITY (LVT Spindle #4, | 3,800 | 5,700 | 9,300 | 15,300 |
| | 12 rpm, 25 C.) | | | | |
| | VISCOSITY (RVF TD Spindle, | — | — | — | — |
| | 12 rpm, 25 C.) | | | | |
| | TEXTURE | 3 | 3 | 3 | 3 |

TABLE 3-2

| | | Example | Example | Example | Example |
|---|---|---|---|---|---|
| | Ingredients | 8 | 9 | 10 | 11 |
| OIL PHASE | Disteardimonium Hectorite | 0.4 | 0.4 | 0.4 | 0.4 |
| | Dimethicone | 13.5 | 9.5 | 9.5 | 7.5 |
| | Phenyl Trimethicone | 4 | 3 | 3 | 3 |
| | PPG-3 Benzyl Ether Myristate, | 2 | 1.5 | 1.5 | 1.5 |
| | Pentaerythrityl Tetra-Di-T-Butyl | | | | |
| | Hydroxyhydrocinnamate | | | | |
| | Trimethylsiloxysilicate, | 4 | 3 | 3 | 2.5 |
| | Cyclopentasiloxane | | | | |
| | PEG-10 Dimethicone, Tocopherol | 2.6 | 2.1 | 2.1 | 2.1 |
| | Bis-ButylDimethicone Polyglyceryl-3 | 1.5 | 1 | 1 | 1 |
| | Sorbitan Sesquiisostearate | 1 | 1 | 1 | 1 |
| | Polymethylsilsesquioxane | 3 | 2 | 2 | 1.5 |
| POWDER | Boron Nitride, Dimethicone | 2 | 1 | 1 | 0.2 |
| | Titanium Dioxide, Mica, Tin Oxide | 1 | 1 | 1 | 1.5 |
| | Mica, Barium Sulfate, Titanium | 1 | 1 | 1 | 1 |
| | Dioxide, Alumina | | | | |
| | Titanium Dioxide, Aluminum | 13 | 9 | 3 | 1.5 |
| | Dimyristate, Triethoxycaprylylsilane, | | | | |
| | Disodium Stearoyl Glutamate | | | | |
| | Iron Oxide, Aluminum Dimyristate, | 7 | 5 | 1.5 | 0.5 |
| | Triethoxycaprylylsilane, Disodium | | | | |
| | Stearoyl Glutamate | | | | |
| TEXTURIZER | Dimethicone, Dimethicone | 10 | 5 | 5 | 2.5 |
| | Crosspolymer | | | | |
| WATER | O/W EMULSION (Example A) | 30 | 50 | 60 | 70 |
| PHASE | Glycerin | 1 | 1 | 1 | 1 |
| | Water | 2.5 | 3 | 2.5 | 1.8 |
| | Phenoxyethanol | 0.5 | 0.5 | 0.5 | 0.5 |
| | Total | 100 | 100 | 100 | 100 |
| PROPERTIES | VISCOSITY (LVT Spindle #4, | 28,900 | — | — | — |
| | 12 rpm, 25 C.) | | | | |
| | VISCOSITY (RVF TD Spindle, | — | 74,800 | 65,300 | 82,700 |
| | 12 rpm, 25 C.) | | | | |
| | TEXTURE | 3 | 3 | 3 | 3 |

The composite emulsions (Examples 4 to 11) had excellent properties similar to those of Example 1. It became apparent that the viscosity of the resultant O/W/O composite emulsion could be adjusted by changing the amount of O/W emulsion incorporated into the inner phase.

O/W/O composite emulsions were prepared with the formulas indicated in Table 4 below. The properties of the composite emulsions were evaluated as in Examples 1 to 3.

TABLE 4

| | | | Comparative | Comparative |
|---|---|---|---|---|
| | Ingredients | Example 12 | Example 1 | Example 2 |
| OIL PHASE | Disteardimonium | 0.4 | 0.4 | 0.4 |
| | Hectorite | | | |
| | Dimethicone | 17.5 | 17.5 | 17.5 |
| | Phenyl Trimethicone | 6 | 6 | 6 |
| | PPG-3 Benzyl Ether | 2 | 2 | 2 |
| | Myristate, Pentaerythrityl | | | |
| | Tetra-Di-T-Butyl | | | |
| | Hydroxyhydrocinnamate | | | |
| | Trimethylsiloxysilicate, | 4 | 4 | 4 |
| | Cyclopentasiloxane | | | |
| | PEG-10 Dimethicone, | 2.6 | 2.6 | 2.6 |
| | Tocopherol | | | |
| | Bis-ButylDimethicone | 1.5 | 1.5 | 1.5 |
| | Polyglyceryl-3 | | | |
| | Sorbitan | 1 | 1 | 1 |
| | Sesquiisostearate | | | |
| | Polymethylsilsesquioxane | 3 | 3 | 3 |
| POWDER | Boron Nitride, | 2 | 2 | 2 |
| | Dimethicone | | | |
| | Titanium Dioxide, Mica, | 1.5 | 1.5 | 1.5 |
| | Tin Oxide | | | |
| | Mica, Barium Sulfate, | 1 | 1 | 1 |
| | Titanium Dioxide, | | | |
| | Alumina | | | |
| | Titanium Dioxide, | 13 | 13 | 13 |
| | Aluminum Dimyristate, | | | |
| | Triethoxycaprylylsilane, | | | |
| | Disodium Stearoyl | | | |
| | Glutamate | | | |
| | Iron Oxide, Aluminum | 7 | 7 | 7 |
| | Dimyristate, | | | |
| | Triethoxycaprylylsilane, | | | |
| | Disodium Stearoyl | | | |
| | Glutamate | | | |
| TEXTURIZER | Dimethicone, | 15 | 15 | 15 |
| | Dimethicone | | | |
| | Crosspolymer | | | |
| WATER | O/W emulsion | 10 | 0 | 0 |
| PHASE | (Example A) | | | |
| | O/W emulsion | 0 | 0 | 10 |
| | (lager than 200 nm) *1) | | | |
| | Deionized Water | 12 | 12 | 12 |
| | Phenoxyethanol | 0.5 | 0.5 | 0.5 |
| | | 100 | 100 | 100 |
| PROPERTIES | RADIANCE | 5 | 3 | 3 |
| | LONG WEAR & | 5 | 3 | 3 |
| | MAKEUP ADHESION | | | |
| | COVERAGE | 5 | 5 | 5 |
| | SKINCARE BENEFITS | 5 | 3 | 3 |
| | (IMMEDIATE) | | | |
| | SKINCARE BENEFITS | 5 | 3 | 3 |
| | (LONG TERM) | | | |
| | TEXTURE | 5 | 3 | 1 |

*1) This is O/W emulsion having a formula identical to that of Example A but having an average particle size of lager than 200 nm.

Example 12, which contains the O/W emulsion of Example A, had excellent properties as Example 1. In contrast, Comparative Example 1, which did not contain an O/W emulsion in its inner phase, was insufficient in “RADIANCE”, “LONG WEAR & MAKEUP ADHESION”, “SKINCARE BENEFITS (IMMEDIATE)”, “SKINCARE BENEFITS (LONG TERM)” and “TEXTURE”. Comparative Example 2, which contains an O/W emulsion having oil particles with average diameter of lager than 200 nm, had further uncomfortable texture.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. A makeup cosmetic, comprising:
an oil-in-water-in-oil composite emulsion in which a water phase comprising an oil-in-water emulsion is dispersed in an external oil phase;
wherein the oil-in-water emulsion, further comprises:
oil droplets having a number-average particle size of 200 nm or smaller; and
said oil droplets are a solidified oil.

2. The makeup cosmetic, according to claim 1, wherein:
the water phase has a number-average particle size of 1 to 10 μm.

3. The makeup cosmetic, according to claim 1 or 2, wherein:
the oil-in-water emulsion constituting the water phase is obtained by emulsification using an anionic surfactant.

4. The makeup cosmetic, according to claim 2, wherein:
the oil-in-water emulsion constituting the water phase is obtained by emulsification using an anionic surfactant.

5. The makeup cosmetic, according to claim 1, wherein:
the content of powder ingredients is 1.0 to 50.0% by mass.

6. The makeup cosmetic, according to claim 2, wherein:
the content of powder ingredients is 1.0 to 50.0% by mass.
